(19) United States
(12) Patent Application Publication
(10) Pub. No.: US 2022/0104876 A1
(43) Pub. Date: Apr. 7, 2022

(54) MICROWAVE APPARATUS AND METHOD

(71) Applicant: Emblation Limited, Alloa (GB)

(72) Inventors: Gary Beale, Alloa (GB); Eamon McErlean, Alloa (GB); Matthew Kidd, Alloa (GB); Shailesh Joshi, Alloa (GB)

(73) Assignee: Emblation Limited, Alloa (GB)

(21) Appl. No.: 17/426,553

(22) PCT Filed: Jan. 29, 2020
(86) PCT No.: PCT/GB2020/050204

§ 371 Date: Jul. 28, 2021

(30) Foreign Application Priority Data
Jan. 29, 2019 (GB) 1901211.1

(51) Int. Cl. A61B 18/18

(52) U.S. Cl. CPC A61B 18/1815; A61B 2018/00023

(57) ABSTRACT

A microwave antenna apparatus comprises a radiating element configured to perform a treatment, the treatment comprising heating a volume of tissue using microwave radiation emitted from the radiating element; a cooling structure arranged for flowing a coolant through at least one lumen of the cooling structure during the treatment; and a controller configured to control the flow of coolant such as to provide a first flow rate of coolant through the at least one lumen during a first period of the treatment and to provide a second, different flow rate of coolant through the at least one lumen during a second, later period of the treatment.

### FIELD

The present disclosure relates to a microwave antenna apparatus or applicator, a microwave assembly, a fluid control system and a microwave system for use in radiating microwave energy into tissues, for example diseased tissues. The present disclosure also relates to methods of controlling the ablation zone of a microwave antenna apparatus or applicator.

### BACKGROUND

Microwave ablation (MWA) technology is a commercially available and popular modality to heat tissue for the treatment of diseased tissues. At microwave frequencies (for example, 915 MHz to 20 GHz), oscillating electromagnetic (EM) fields are utilised to create friction in water molecules that leads to heat formation within tissues. Such tissues may include, for example, tumours. Treatments in the hyperthermia (sub 50° C.) or ablative (above 50° C.) regimes may be used. The thermal response in the tumour can act as an adjuvant to other therapies such as chemotherapy and radiotherapy.

Alternatively, MWA may be used to kill tissues in situ when removal or resection is clinically complex or presents high risk for the candidate. Tumours may be present in tissues within, for example, the liver, kidney, lung, bones or any other tissue. A necrosis region resulting from MWA may typically be intended to encapsulate the tumour and a margin of tissue around it to ensure no regrowth or spread of the tumour to other tissues.

MWA may be particularly suited for use in minimally invasive procedures to perform thermal ablation. Minimally invasive procedures for thermal ablation may be applied using one or more needle-like antennae. In order to reach the target tissue with a minimal amount of damage caused by the insertion, a shaft of a needle-like antenna has an elongated aspect ratio. The shaft contains a coaxial transmission line (coaxial cable) capable of delivering sufficient power to heat a target volume. A diameter of the shaft may be optimised to balance having a shaft that is large enough to contain the coaxial transmission line, versus having a compact antenna. In some circumstances, a larger shaft may risk insertion damage or risk of bleeding and pneumothorax.

The antenna form may be, for example, slot, monopole, dipole or triaxial. An efficiency of each antenna form may be tuned to specific microwave frequencies and tissue property types.

It may be desirable to have a predictable pattern of radiation emitted by the antenna and therefore a predictable pattern of subsequent heating. A pattern or shape that may be considered to be of most use to clinicians when planning a procedure may be a spherical pattern.

The simulation of radiation pattern in different tissue types and dimensions is common practice. Simulation of radiation pattern may be verified in ex vivo tissue such as liver as part of a development process.

It has been found that common design of microwave antenna used in a small diameter shaft may typically yield a lachrymiform (tear drop shaped) pattern of radiation and heating. The lachrymiform shape comprises a tail of energy that is found extending back along the cable. As this tail feature may deliver heat to tissues outside the target zone that lead to ablation of healthy tissue, it may be deemed undesirable.

A common method of creating a more spherical radiation pattern may be to use a balun or choke feature that balances the energy delivered along the axis with that delivered perpendicularly. Alternating currents flowing in an antenna element result in an electromagnetic wave being generated and radiated into the surrounding tissue. The currents flowing in the antenna element connected or coupled to the transmission line outer conductor have to return to the generator and achieve this by flowing on the outer conductor of the coaxial transmission line. Currents returning by flowing on the outer conductor may be referred to as common mode currents. These common mode currents induce radiation from the outer conductor of the coaxial transmission line which can distort the antenna radiation pattern.

However, the addition of balun or choke features may lead to a loss of overall antenna efficiency, increase the overall shaft diameter and/or add complexity to a manufacture process.

A challenge to achieving optimum ablation volume capacity may be the transfer of power from the microwave generator to the antenna. Coaxial cables are most commonly used due to their mechanical flexibility and transverse electric and magnetic wave propagation. The power handling of coaxial cables is directly related to their size and choice of materials. Attenuation losses within a coaxial cable are measured in loss per length e.g. dB/m. Attenuation losses are exhibited as heat. Typically, higher operating frequencies and smaller diameters increase losses.

Table 1 shows loss values for two different coaxial cables. SUCOFORM_47_CU has an outer diameter of 1.194 mm. SUCOFORM_86 has an outer diameter of 2.19 mm. The loss values in Table 1, and in Table 2 below, are taken from Microwave cable assemblies, page 161, Edition 2019/0,1 HUBER+SUHNER AG. The loss values in Table 1 are provided for different frequencies at a constant temperature and pressure for the two different coaxial cables.

TABLE 1

Loss (dB/m) at +25° C. ambient
temperature and sea level

| Diameter (mm) | 2.5 GHz | 12 GHz |
|---|---|---|
| SUCOFORM_47_CU 1.194 | 1.890 | 4.400 |
| SUCOFORM_86 2.159 | 1.093 | 2.657 |

The power handling ability of a cable to safely transfer power without overheating or failure, may be related to the same loss factors, for example frequency of the applied microwave power and cable diameter.

Table 2 shows power handing for different frequencies at a constant temperature and pressure for the two different coaxial cables.

TABLE 2

| | Power handling (W) at +40° C. ambient | |
|---|---|---|
| | temperature and sea level | |
| Diameter (mm) | 2.5 GHz | 10 GHz |
| SUCOFORM_47_CU 1.194 | 20 | 9 |
| SUCOFORM_86 2.159 | 102 | 51 |

The demand for increased power to treat larger regions and improve treatment times may lead to the choice being made to cool the coaxial cable to prevent heat damage to tissue adjacent to the probe shaft. Cooling the coaxial cable may be used to maintain the cable integrity to prevent failure from material breakdown.

Cooling of coaxial cables may be achieved in a number of ways. Currently, gas and fluid methods dominate the mediums used for cooling. A gas such as air or other cryogenic substance may be contained within a lumen the length of the shaft of the antenna. The flow and return of the gas may be made with a series of lumens in order to maintain heat transfer for the duration of the antenna operation. Similarly, in some cases, liquids such as saline travel the length of the cable in the shaft through one or more lumens.

The flow of the cooling medium may commonly be achieved through the use of a pump. Temperature sensors may be placed along the length of the shaft to provide data to ensure the system is operating within specified safe temperature bounds. The pump is typically operated at a single constant setting to provide a flow rate that prevents exceeding upper temperature bounds in the extreme case of maximum power for maximum duration in an ambient environment at the limit of system operation specification.

### SUMMARY

One of ordinary skill in the art should understand that any of the features of any one of the apparatus, assemblies, systems or methods described herein may apply alone or in any combination in relation to any other one of the apparatus, assemblies, systems or methods described herein.

A microwave antenna apparatus and cooling system is described herein for use in radiating microwave energy into tissues for the purpose of heating. A method of achieving the desired ablation volume geometry to achieve optimal clinical outcomes is proposed. By controlling the flow of the cooling medium, heating in the proximal region of an antenna can be influenced. The flow rate of the cooling medium can be adjusted by controlling a pump speed of a pump, for example a peristaltic pump. Flow rates may be, but are not limited to, 0.1 to 100 ml/min, 10 to 110 ml/min, 15 to 200 ml/min, 20 to 400 ml/min, 30 to 500 ml/min.

In the case of an antenna where there is emission of radiation along the shaft proximal to the target heating zone, a certain flow rate of cooling medium can be used to counteract the heating effect and therefore achieve a more symmetrical and spherical heating zone. When the rate of flow is low, it is found that there may be a heat affected region outside the desired ablation sphere. When the rate of flow is sufficiently high, heat may be removed within the desired ablation sphere, thereby leaving diseased tissue under-treated. It is found that there exists a combination of flow rates where neither of the negative effects of undercooling or overcooling takes place i.e. the hybrid heat flow is balanced. This flow rate may preferentially be modulated during the treatment.

An embodiment of modulation is to have different flow rates for different periods of the treatment such that the start of the treatment is deemed low flow rate and then operated at a high flow rate. Conversely, an alternative embodiment would be to start with a high flow rate and then operate at a low flow rate towards the end of the treatment. A further embodiment would be to have a flow rate profile that varied over time such that a sequence of flow rates were maintained for varying periods of time. Modulating the flow rate appropriately to control the presence of heating outside the ablation sphere or other desired volume may permit a more compact, simpler antenna design and/or allow a higher delivery of power.

An embodiment that biases the flow of coolant asymmetrically is proposed. Rather than concentric lumens of flow and return for the coolant, there may be multiple lumens around a central cavity for the coaxial cable. Cooling may be biased to one side of the overall system as the flow from the coolant source is at a lower temperature than the return flow that has been heated at the main antenna radiating point.

A further embodiment may use the same heat controlling effect asymmetrically to the antenna such that one side of the ablation zone proximal to the generator is preferentially cooled greater than the side diametrically opposite to the generator relative to the antenna shaft. An asymmetry may exist on either side of the main axis of the shaft, when looking along the main axis of the shaft. A region on one side of the main axis may be preferentially cooled.

This effect may have an advantage where sensitive tissues such as arteries, veins or nerves may be present immediately adjacent to the target treatment zone and the shaft of the antenna. This embodiment may also be used when the target tissue and surrounding tissue are of different dielectric properties such that the antenna tuning results in an asymmetric ablation geometry and the bias of the heat affected zone along the shaft is outside the desired zone. Here, the asymmetry of the flow diametrically opposite of the shaft can be utilised to either protect non-target tissue or conversely, assist in expanding the heated zone to match the desired volume.

The design of the flow and return lumens for the coolant in a concentric lumen arrangement may also be utilised. When the coolant is supplied and the flow is contained in the outer lumen the cooling effects are, for the same geometry, different from the alternative operation then the flow is in the inner lumen, contacting the coaxial cable.

The choice of flow lumen may be advantageous when seeking to protect the tissues the shaft is in contact with. For example, when the shaft is intra cavity of an artery, vein, urethra or other natural lumen, one may seek to protect the tissues of the natural lumen. When the outer lumen is used for the flow, the external shaft temperatures are typically lower than when the return flow is in the outer lumen. This may permit an ablation to be delivered whilst preserving the natural lumen, or using the lumen as an access point to treat an area that may be adjacent to the natural lumen which could be damaged under normal circumstances due to its proximity to the ablation zone.

An embodiment of flow rate varying over time (for example in a 10 minutes of total treatment time, low flow for 2 minutes followed by high flow for 8 minutes to maintain a spherical ablation with no lachrymiform feature) may also be influenced by flow lumen choice. Depending on the geometry of the lumens, the flow rates necessary to achieve the desired shape may be significantly different. This may be advantageous when the design of the system is being made and the capacity of the coolant pump is chosen.

In a first aspect of the invention, there is provided a microwave antenna apparatus comprising: a radiating element configured to perform a treatment, the treatment comprising heating a volume of tissue using microwave radiation emitted from the radiating element; a cooling structure arranged for flowing a coolant through at least one lumen of the cooling structure during the treatment; and a controller configured to control the flow of coolant such as to provide a first flow rate of coolant through the at least one lumen during a first period of the treatment and to provide a second, different flow rate of coolant through the at least one lumen during a second, later period of the treatment.

The controller may be configured to control the flow of coolant such as to shape the volume of tissue that is heated by the radiating element during the treatment.

The controller may be configured to control the flow of coolant such as to form in the tissue a spherical treatment zone. The controller may be configured to control the flow of coolant such as to form in the tissue a hybrid spherical lachrymiform treatment zone. The controller may be configured to control the flow of coolant such as to form in the tissue a partial lachrymiform treatment zone. The controller may be configured to control the flow of coolant such as to form in the tissue a partial spherical treatment zone.

The controller may be configured to control the flow of coolant such that the first flow rate during the first period is higher than the second flow rate during the second period.

The controller may be configured to control the flow of coolant such that the first flow rate during the first period is lower than the second flow rate during the second period.

The first period may be longer than the second period. The second period may be longer than the first period.

The controller may be configured to vary the flow rate continuously over time during the treatment.

The controller may be further configured to alter a power of the radiation emitted from the radiating element during the treatment.

The controller may be configured to control the power of the radiation such as to shape the volume of tissue that is heated by the radiating element during the treatment.

The cooling structure may comprise at least one inner lumen positioned radially proximal to the radiating element and at least one outer lumen positioned radially distal to the radiating element.

The controller may be configured to control the flow of coolant such that the coolant flows into the at least one inner lumen and returns through the at least one outer lumen.

The controller may be configured to control the flow of coolant such that the coolant flows into the at least one outer lumen and returns through the at least one outer inner.

The cooling structure may comprise a plurality of lumens arranged circumferentially around the radiating element.

The controller may be configured to select lumens through which the coolant flows in and out such as to shape the volume of tissue that is heated by the radiating element during the treatment.

At least one diameter of at least one lumen may be selected such as to shape the volume of tissue that is heated by the radiating element during the treatment.

The shape of the volume of tissue that is heated may be controlled so as to reduce or eliminate heating of an anatomical structure. The anatomical structure may comprise at least one natural lumen.

The shape of the volume of tissue that is heated may be controlled to reduce tissue shrinkage in the vicinity of the radiating element.

The apparatus may further comprise at least one temperature sensor configured to monitor temperature within the volume of tissue to be heated.

The controller may be configured to control the flow of coolant in dependence on signals from the at least one temperature sensor, wherein the signals are representative of the monitored temperature.

The controller may be further configured to control in dependence on the signals from the at least one temperature sensor at least one of: a power supplied to the radiating element, a direction of coolant flow, a coolant flow path.

The radiating element may be formed from coaxial cable.

The radiating element may comprise at least one of a slot antenna, a monopole antenna, a dipole antenna, a triaxial antenna.

The coolant may comprise at least one of a gas coolant, a liquid coolant, air, saline.

The apparatus may be configured to perform microwave ablation of tissue and/or tissue hyperthermia.

The flowing of the coolant through at least one lumen of the cooling structure may be to cool the radiating element and/or to cool a cable supplying power to the radiating element.

The radiating element and the cooling structure may be housed within a common housing.

In a further embodiment, there is provided a microwave system comprising: a microwave generator; a microwave cable apparatus comprising a coaxial cable, wherein an exposed distal portion of an inner conductor of the coaxial cable is longer than an outer conductor of the coaxial cable, the exposed distal portion forming a radiating element, wherein the radiating element is configured to perform a treatment, the treatment comprising heating a volume of tissue using microwave radiation emitted from the radiating element; a cooling structure arranged for flowing a coolant through at least one lumen of the cooling structure during the treatment; and a controller configured to control the flow of coolant such as to provide a first flow rate of coolant through the at least one lumen during a first period of the treatment and to provide a second, different flow rate of coolant through the at least one lumen during a second, later period of the treatment.

In a further aspect, there is provided a method comprising: performing a treatment comprising heating a volume of tissue using microwave radiation emitted from a radiating element; during the treatment, flowing a coolant through at least one lumen of a cooling structure; and controlling the flow of coolant such as to provide a first flow rate of coolant through the at least one lumen during a first period of the treatment and to provide a second, different flow rate of coolant through the at least one lumen during a second, later period of the treatment.

The controller may be configured to control the flow of coolant such as to shape the volume of tissue that is heated by the radiating element during the treatment.

In a further aspect, there is provided a method comprising: receiving parameters of a radiating element for emission of microwave radiation and a cooling structure comprising at least one lumen; receiving a desired volume of tissue to be heated by the emission of microwave radiation from the radiating element; and determining a first flow rate of coolant and second flow rate of coolant to be provided through the at least one lumen to shape a volume of tissue heated by the emission of microwave radiation by the radiating element to match the desired volume of tissue, wherein the determining is in dependence on the parameters of the radiating element and the cooling structure.

The method may further comprise determining a direction of coolant flow through the at least one lumen. The method may further comprise determining a path of coolant flow. The method may further comprise determining a power to be provided to the radiating element. The method may further comprise determining a period of time over which the first flow rate is to be delivered. The method may further comprise determining a period of time over which the second flow rate is to be delivered.

The determining may be further in dependence on at least one temperature measurement for tissue heated by the radiating element.

In a further aspect of the invention, there is provided a method of shaping the treatment volume of a microwave antenna by controlled use of coolant flow rate contained within the applicator. The controlled use of coolant flow rate may comprise a hybrid of low-flow and high-flow. The controlled use of coolant flow rate may comprise a main proportion of high flow with a minor proportion of low flow. The controlled use of coolant flow rate may comprise a main proportion of low flow with a minor proportion of high flow. The controlled use of coolant flow rate may comprise a continuously varied flow rate. The controlled use of coolant flow rate may comprise a time number of different constant flow rates. The controlled use of coolant flow rate may comprise a time number of different varied flow rates.

The method may comprise applying fixed or varied power.

The shaping of the treatment volume may be to create a spherical treatment zone. The shaping of the treatment volume may be to create a hybrid spherical lachrymiform treatment zone. The shaping of the treatment volume may be to create a partial lachrymiform treatment zone. The shaping of the treatment volume may be to create a partial spherical treatment zone. Although, spherical treatment zone maybe the preferred type, the shaping of the treatment volume in the present invention may be used to create any other desired shape treatment zones for example but not limited to total or partial elliptical, hybrid elliptical lachrymiform where elliptical shape may be prolate or oblate.

The controlled use of coolant means may comprise a constant flow rate to achieve a desired pattern for a given power and duration. The controlled use of coolant means may comprise varied flow rate to achieve a desired pattern for a given power and duration.

A coolant lumen dimension may be chosen to bias the flow rate to achieve a desired pattern for a given power and duration. A coolant flow direction may be chosen to bias the flow rate to achieve a desired pattern for a given power and duration. A coolant lumen dimension may be chosen to bias the flow rate to achieve a desired pattern for a given power and duration. A number and arrangement of coolant lumens may be chosen to bias the flow rate to achieve a desired pattern for a given power and duration.

Controlled use of coolant means may be used to bias a desired pattern to reduce proximity to a natural lumen.

In a further aspect of the invention, there is provided an algorithm to control the power and coolant flow rate contained within an applicator to deliver a prescribed ablation zone.

In a further aspect of the invention, there is provided an algorithm to control the power, coolant flow rate and temperature within an applicator to deliver a prescribed ablation zone.

In a further aspect of the invention, there is provided an algorithm to monitor the power, coolant flow rate and temperature within an applicator to deliver a prescribed ablation zone

In a further aspect of the invention, there is provided an algorithm to monitor an ablation zone and to adjust the power and coolant flow rate contained within an applicator to deliver a prescribed ablation zone.

In a further aspect of the invention, there is provided an algorithm to monitor the antenna temperature and to adjust the power and coolant flow rate contained within an applicator to deliver a prescribed ablation zone.

In a further aspect of the invention, there is provided a method of applying a hybrid cooling profile to reduce overall tissue shrinkage in proximity to the ablation probe during an ablation.

In a further aspect of the invention, there is provided use of coolant control to permit use of an antenna in a natural lumen to preserve the lumen.

Features in one aspect may be applied as features in any other aspect, in any appropriate combination. For example, system features may be provided as method 15 features or vice versa.

### BRIEF DESCRIPTION OF THE DRAWINGS

Embodiments of the invention are now described, by way of non-limiting examples, and are illustrated in the following figures, in which:—

FIG. 1A is a schematic illustration of a microwave system;

FIG. 1B is an illustration of a coaxial cable/transmission line construction/assembly;

FIG. 2A is an illustration of a microwave treatment system in accordance with an embodiment;

FIG. 2B is a detailed cross-sectional view of the microwave treatment system of FIG. 1;

FIG. 3 is an illustrative of a typical shape of an electromagnetic specific absorption rare (SAR) pattern created by a simple monopole antenna contained within coolant lumens;

FIG. 4 is an illustration of the tissue necrosis created theoretically using a simple monopole antenna of the microwave system of FIG. 1 with low flow rate in accordance with an embodiment;

FIG. 5 is an illustration of the tissue necrosis created theoretically using the microwave treatment system of FIG. 1 comprising a cooling system having a modulated flow rate from high to low;

FIG. 6 is an illustration of the tissue necrosis created theoretically using the microwave treatment system of FIG. 1 comprising a cooling system having a modulated flow rate from low to high;

FIG. 7 is an illustration of the tissue necrosis created theoretically using the microwave treatment system of FIG. 1 comprising a cooling system having a modulated flow rate from low to high and inverted flow direction;

FIG. 8 is an illustration of the tissue necrosis created theoretically using the microwave treatment system comprising a cooling system having an optimised modulated flow rate from low to high and inverted flow direction;

FIG. 9 includes schematic illustrations 9(a) to 9(e) of treatment profiles entailing various combinations of high and low flow rate and various durations;

FIG. 10 is an illustration of the tissue necrosis created theoretically using the microwave treatment system of FIG. 1 comprising an inverted cooling system with an increasing modulated flow rate for creating a certain ablation shape;

FIG. 11 is a photograph of ex vivo bovine liver tissue ablation created using a prototype of a microwave treatment system at a low power and a high coolant flow rate;

FIG. 12 is a photograph of ex vivo bovine liver tissue ablation created using a prototype of a microwave treatment system at a high power and a high coolant flow rate; and

FIG. 13 is a photograph of ex vivo bovine liver tissue ablation created using a prototype of a microwave treatment system at a low power and a low coolant flow rate.

### DETAILED DESCRIPTION

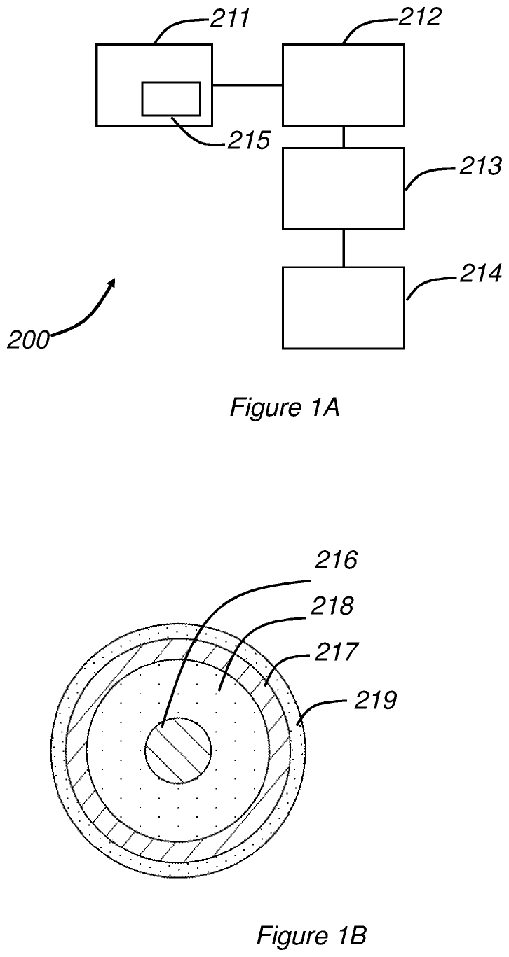

FIG. 1A illustrates a microwave system generally designated 200 for treating a tissue. The microwave system 200 comprises a microwave generator 211 for providing microwave energy, a flexible interconnecting microwave cable such as a coaxial cable 212, a hand grip or hand piece 213, and a microwave antenna apparatus 214. The microwave generator 211 comprises a controller 215 configured to select a frequency of microwave energy provided to the cable apparatus and/or a power of microwave energy provided to the cable apparatus.

FIG. 1B is a cross-sectional illustration of a coaxial cable that may be used as a flexible interconnecting microwave cable 212 in the system of FIG. 1A. The coaxial cable may also be referred to a transmission line. The construction of a typical transmission line (coax) shown in FIG. 1B includes a flexible coaxial transmission line (coax) including a flexible centre conductor 216 coaxial with a flexible cylindrical outer conductor 217. An insulating material 218 substantially fills the space between centre conductor 216 and outer conductor 217. The insulating material 218 may also be referred to as a dielectric material. The insulating material 218 is for holding the centre conductor 216 and outer conductor 217 in place and for electrically isolating the conductors from each other.

The outer conductor 217 may be referred to as a primary outer conductor. The primary outer conductor 217 may be augmented with a second flexible conductive sheath or braid (not shown), which may be positioned outside the primary outer conductor 217.

In turn, the outer conductor 217 or the second flexible conductive sheath or braid may be coated over its length by a flexible jacket 219. The flexible jacket 219 may be made of an inert impermeable and low friction material, for example FEP (Fluorinated ethylene propylene). A suitable type of coaxial transmission line is manufactured by HUBER+SUHNER (Switzerland) reference by type SUCOFORM_43_FEP_MED having nominal outer FEP jacket diameter of 1.09 mm, a dielectric diameter of 0.84 and flexible centre conductor diameter of 0.31 mm.

In other embodiments, other coaxial transmission lines may be used, for example coaxial transmission lines having different dimensions and/or formed from different materials. In some embodiments, the coaxial cable may be semi-rigid or rigid.

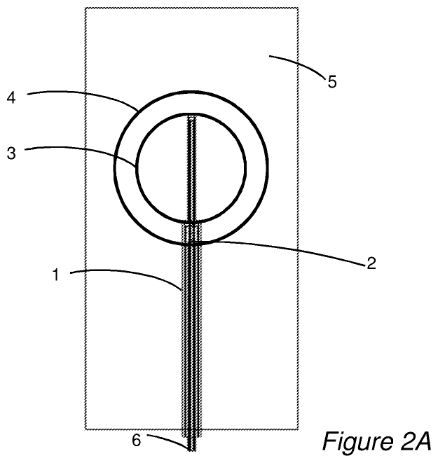

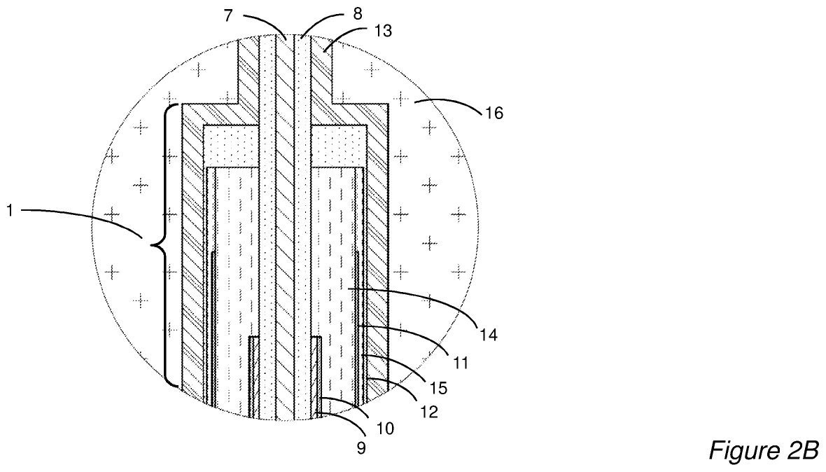

A diagrammatic illustration of a microwave ablation antenna 1 is shown in FIG. 2A. The microwave ablation antenna 1 is formed from a coaxial cable 6, for example from a coaxial cable as described above with reference to FIG. 1B. The antenna may be formed, for example, by cutting an outer conductor of the coaxial cable 6 thus exposing part of the inner conductor of the coaxial cable to form a monopole antenna. At the distal end of an outer conductor of the coaxial cable 6 the monopole antenna may be considered to start at 2. The monopole antenna may also be referred to as a radiating element.

In use, the microwave ablation antenna 1 is coupled to a microwave generator 211 (not shown in FIG. 2A). The monopole antenna is placed near to or contacting the tissue of a patient. The microwave generator 211 generates microwave energy and supplies microwave energy to the microwave ablation antenna 1. At least some of the supplied microwave energy is radiated from the monopole antenna into the tissue of the patient.

In the scenario shown in FIG. 2A, the monopole antenna is placed inside a tumour 3 that is to be treated by microwave ablation. The microwave energy treats the tumour 3 along with a margin 4 that sits in the healthy tissue 5 surrounding the tumour 3.

FIG. 2B is a cross-sectional view of a microwave ablation antenna in accordance with an embodiment. The detail of an example embodiment of antenna with coolant and cable interface is shown in FIG. 2B where the coaxial cable is contained within lumens 11 and 12 allowing the flow coolant 14 to return as 15. The lumens 11, 12 and coaxial cable are positioned within the optional shaft 13 which is configured to be inserted into diseased tissue 16. The monopole antenna of the microwave ablation antenna is formed from the inner conductor 7 of a coaxial cable comprising the coaxial components of inner conductor 7, dielectric 8, outer conductor 9, outer jacket 10. The microwave ablation antenna 1 may not be limited to monopole designs and may be but not limited to slot, dipole or triaxial antenna types.

In the embodiment of FIG. 2B, the monopole antenna is formed by cutting back the outer conductor 9 and outer jacket 10 of the coaxial cable. The dielectric 8 is retained along at least part of the exposed inner conductor 7.

A first lumen 11 surrounds the coaxial cable and a part of the exposed monopole antenna.

The first lumen forms a concentric cylinder around the coaxial cable and the part of the exposed monopole antenna. The first lumen may be referred to as an inner lumen. The first lumen defines a first cooling channel 14 for coolant flow.

A second lumen 12 forms a further concentric cylinder surrounding the first lumen 11. The second lumen 12 defines a second cooling channel 15 for coolant flow. The second cooling channel 15 is concentric with the first cooling channel 14 and is positioned outside the first cooling channel.

The first lumen 11 and second lumen 12 together form a cooling structure. In the present embodiment, the flow of coolant through the cooling structure cools the monopole antenna and the coaxial cable. The shaft provides a housing for the antenna and cooling structure.

The flow of the cooling medium may be achieved through the use of a pump (not shown), for example a peristaltic pump.

Peristaltic pumps may range from basic pumps with minimum control to advanced pumps that have high accuracy and greater control over the pump parameters. Flow rate (speed of the cooling medium) may be controlled manually on the unit. For example, the qdos series by Watson-Marlow can provide flow rates from 0.1 ml/min to 2000 ml/min.

Alternatively, using pumps with control technologies such as Masterflex by Cole-Parmer, pump parameters including speed, flow rate (0.0006 to 3400 mL/min), and dispense volume can be controlled in real-time using a computer, laptop, tablet or phone. Communication from the microwave generator may be achieved by analogue or digital (PWM, SPI, USB, Serial) or PLC remote control, SCADA or Profibus network control. In some embodiments, low rates may be, for example, 0.1 to 100 ml/min, 10 to 110 ml/min, 15 to 200 ml/min, 20 to 400 ml/min, or 30 to 500 ml/min.

In the present embodiment, coolant flowing outward from the pump into the antenna flows through the first cooling channel 14 within the first lumen 11, and coolant returning to the pump flows through the second cooling channel 15 within the second lumen 12 (and outside the first lumen 11). The first cooling channel 14 and second cooling channel 15 are in fluid communication at the end of the channels 14, 15 that is distal from the pump (and is positioned by the monopole antenna).

In other embodiments, the pump pumps coolant into the first cooling channel 15, and coolant returns to the pump through second cooling channel 14.

In the present embodiment, the controller 115 controls the operation of the pump, thereby controlling a direction of flow of the coolant and a flow rate of the coolant. In other embodiments, any suitable controller or controllers may control the direction of flow and/or flow rate and/or flow path of the coolant.

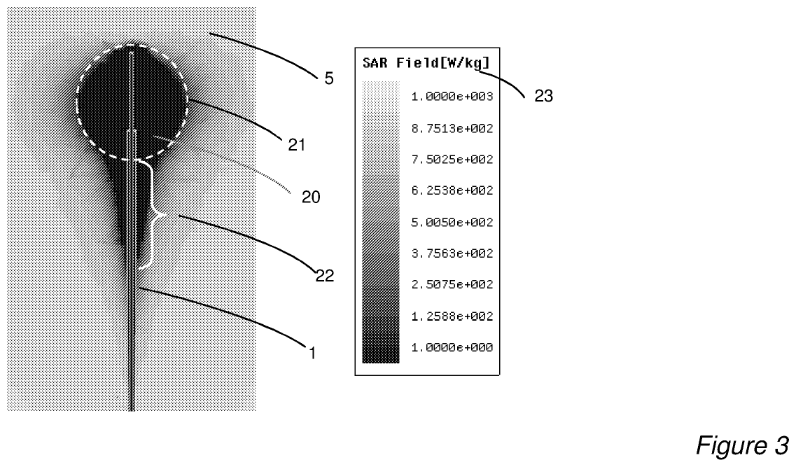

An example of a typical monopole antenna is given as reference in FIG. 3 which has been simulated using a 3D simulation model. In this case, the simulation model is HFSS (Ansoft Corp) which is a Finite Element Method (FEM) based full wave electromagnetic solver. Any appropriate simulation method may be used to simulate antennas according to embodiments. Simulations may allow the calculation of a predicted response for coupling efficiency and specific absorption rate (SAR). SAR is a measure of the rate at which energy is absorbed by the human body when exposed to a radio frequency (RF) electromagnetic field.

In the example of FIG. 3, the monopole antenna is not cooled. HFSS is used to calculate a SAR cross section for the monopole antenna. The SAR cross section predicts the performance of delivering energy into tissue, which may be diseased tissue. In FIG. 3, the monopole antenna delivers energy into a region 20, which appears as a dark area of the SAR cross section plot.

As a reference, a circle 21 illustrates what might be a desirable target shape for radiation delivered by the monopole antenna. It may be desired that the radiation delivered by the monopole antenna is spherical. Instead, in the example shown in FIG. 3, the region 20 in which radiation is delivered is a lachrymiform (tear drop shaped) region having a tail part 22.

The lachrymiform feature 22 that extends along the coaxial cable is an undesirable feature. The legend of the SAR plot 23 can be used to show the level of SAR where 1×10³ W/kg is the maximum in this simulation.

When considering ablation it is informative to consider if the tissue has reached the temperature threshold for performing ablation; a SAR plot cannot report temperature information.

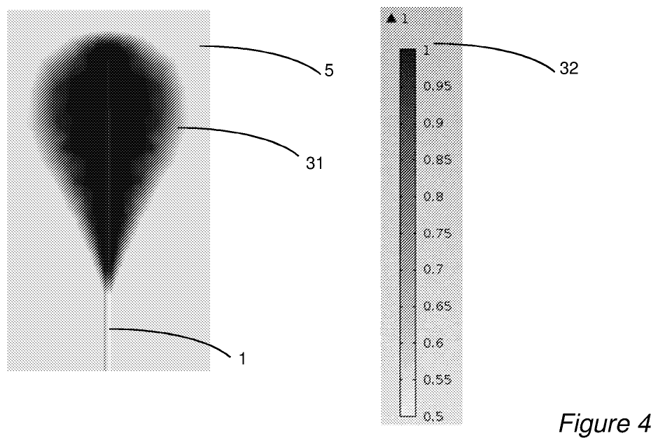

In the simulated performance of the embodiments are necrosis tissue plots such as FIG. 4 where the diagrammatic side view representation of the antenna 1 and region of necrosed tissue 31 was calculated with Comsol (COMSOL AB, Sweden) modelling software which is a Finite Element Method (FEM) solver. The necrosis factor 32 represents treated versus untreated tissue. FIG. 4 shows the necrosis region 31 obtained using a simple monopole antenna cooled by coolant having a low, constant flow rate. The flow was maintained over a treatment time of 10 minutes. The necrosis region 31 has a lachrymiform shape which may be considered to be undesirable.

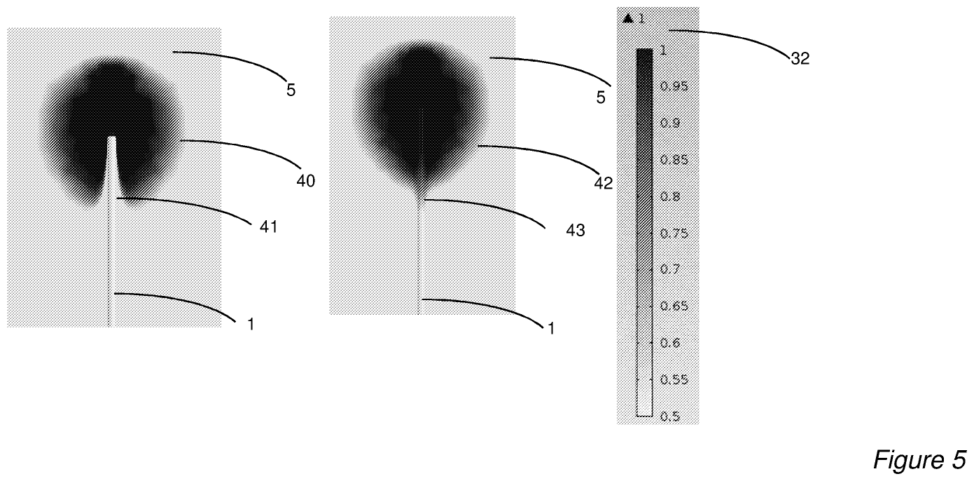

An embodiment that illustrates the impact of high coolant flow rate is shown in FIG. 5. FIG. 5 shows the theoretical necrosis from a cooling system having flow that is modulated from a high flow rate to a low flow rate. Coolant is pumped into an inner jacket-port into the inner lumen 11. The coolant then returns through outer lumen 12. The embodiment may be considered to have a hot outer.

A flow rate of 110 ml/min corresponding to a flow velocity of V=1.78 m/s was maintained for 8 minutes, which was 80% of the treatment time.

The left plot in FIG. 5 shows the region of necrosed tissue 40 obtained at the end of the first 8 minutes, where the spherical form of necrosed tissue is shown by 40 and an under treated region 41 surrounds applicator assembly 1. The high flow rate of coolant used for the first 8 minutes results in a region of under treatment 41 around the antenna, because this region is cooled to a temperature that is too low for necrosis to be achieved.

With the flow rate altered after 8 minutes from 110 ml/min to 12.3 ml/min (V=0.2 m/s), the spherical form 42 is improved as the region 43 begins to extend along the shaft. Such an embodiment may achieve a desirable necrosed region using a hybrid cooling profile. This approach may also have benefit in reducing the compression of tissue around the antenna probe due to shrinkage during treatment which can make it difficult to withdraw the probe causing the antenna to be damaged.

The use of two or more coolant flow rates to control a degree of cooling of the radiating antenna element may provide an efficient method of controlling a volume of tissue treated. The method may be flexible. In some circumstances, different shapes of treatment may be achieved by controlling flow rate without changing the apparatus used. A compact antenna may be produced. It may be possible to avoid adding features such as a balun or choke that may add complexity to a manufacture process.

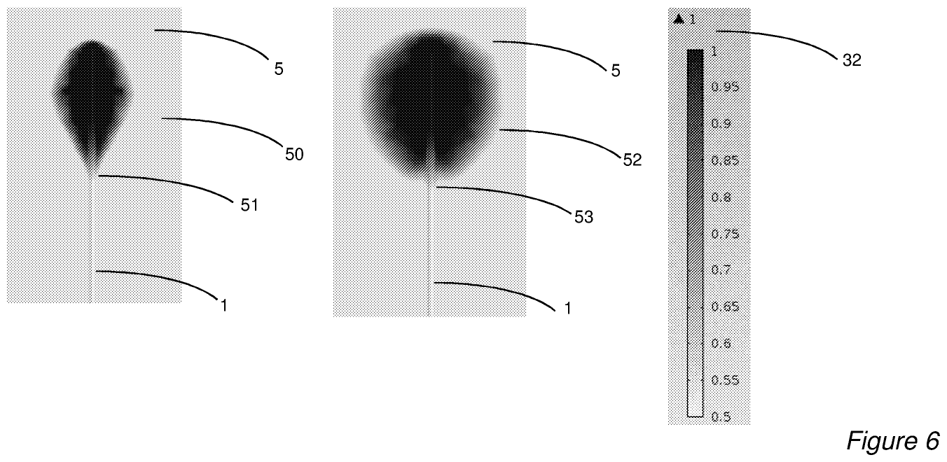

FIG. 6 shows the theoretical necrosis in a further embodiment. A further embodiment of modulated flow in FIG. 6 uses a slower initial flow rate of 12.3 mL/min (V=0.2 m/s) for 2 minutes, increased to 110 mL/min (V=1.78 m/s) for 8 minutes to complete the ablation in the same time of 10 minutes. The left plot of FIG. 6 shows the initial lachrymiform necrosis region 51 achieved in the first 2 minutes. The right plot of FIG. 6 shows the achieving of a circular form 52 in the following 8 minutes, with no lachrymiform feature in the region of 53.

The embodiment of FIG. 6 pumps coolant into the inner lumen 11 and returns coolant through the outer lumen 12.

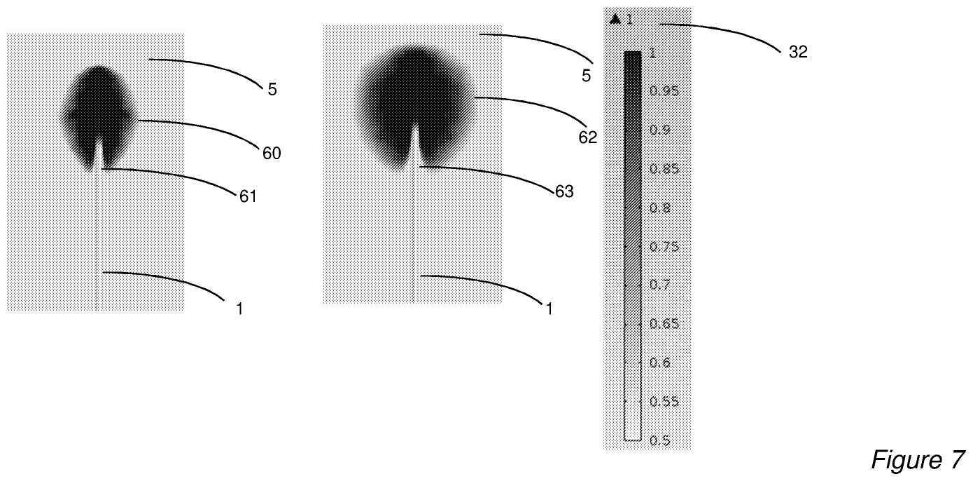

FIG. 7 shows the theoretical necrosis in a further embodiment. The embodiment of FIG. 7 has a reversed coolant lumen configuration of flow and return with reference to that shown in FIG. 2B. Coolant is pumped into the outer lumen 12 through an outer jacket-port (inlet) and returned through the inner lumen 11. A more pronounced under treated region 61 is shown in FIG. 7 which used the same flow rate of 12.3 mL/min (which in this case corresponds to a flow velocity of V=0.57 m/s) and duration of 2 minutes initially (left plot of FIG. 7). A flow rate of 110 ml/min (V=5.12 m/s) is then used for 8 minutes (right plot of FIG. 7). If the sequence used in FIG. 6 is continued with this reversed flow direction, the under-treated region 63 grows along with the treated region 62. It is found that keeping the same flow rate as the inner jacket-port embodiment of FIG. 6 results in ablation with more null zones, suggesting that in this case the flow rate is too high.

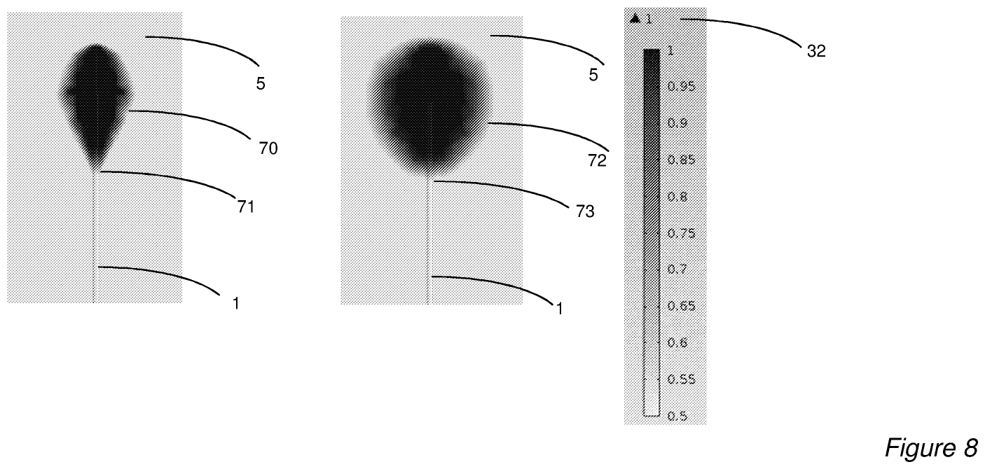

FIG. 8 shows theoretical necrosis for a further embodiment. In order to optimise the flow rate profile to desired circular shape of necrosis the embodiment shown in FIG. 8 is used which maintains the reverse flow direction of the embodiment of FIG. 7. A flow rate of 8 ml/min (V=0.37 m/s) is used for the first 2 minutes (left plot of FIG. 8). A flow rate of 30 ml/min (V=1.4 m/s) is then used for the following 8 minutes (right plot of FIG. 8). Although the form of treated region 70 features the lachrymiform 71 after 2 minutes flowing at a lower flow rate of 8 mL/min, when increasing the flow to 30 mL/min for a subsequent 8 minutes the resultant shape 72 is a desirable circular form. A similar spherical ablation zone 72 is achieved as compared to 52 in FIG. 6 but at a much lower flow rate with no apparent lachrymiform feature in the region of 73. Flow rate is reduced using the outer jacket-port.

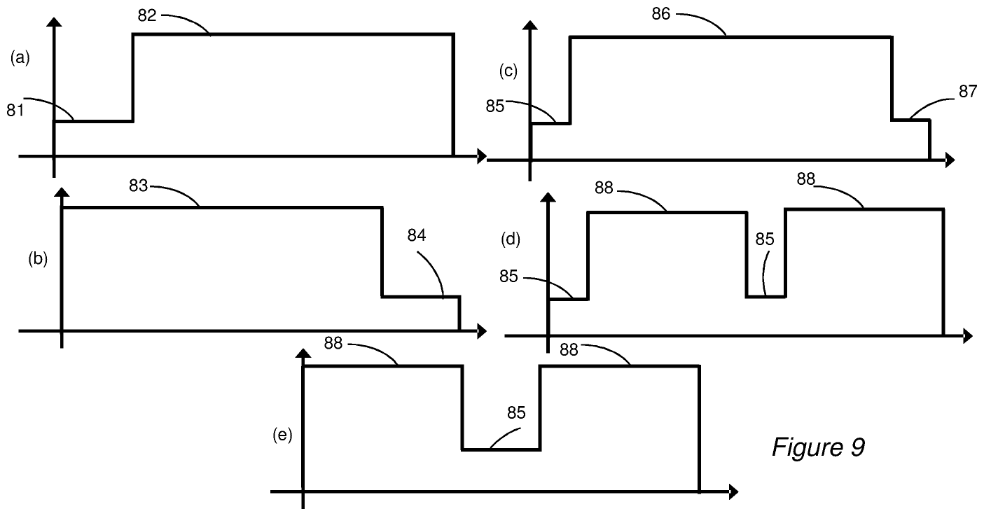

A variety of flow profiles can be considered as further examples of embodiments such as those illustrated in FIG. 9. The flow rate (Y axis) is plotted against time (X axis) for a range of modulated cases.

In FIG. 9(a) a low initial flow rate for a short duration 81 as a proportion of the whole is followed by a high flow rate for a long duration 82. The inverse case of FIG. 9(a) is given in FIG. 9(b) where a high initial flow rate for a long duration 83 is followed by a low flow rate for a short duration 84 at the end of the procedure.

Further examples of embodiments are given with a more complex sequence such as FIG. 9(c) where the main duration of high flow rate 86 is preceded 85 and succeeded 87 by short durations of low flow rate. A repetition of sequence is shown in FIG. 9(d) where both low flow rate and short durations 85 are combined with medium durations of high flow rate 88. FIG. 9(e) shows an interrupted high flow medium duration 88 with a low flow rate for short duration 85. The flow profile could be part of an algorithm which builds a selected ablation zone based upon control of flow and control of power delivered to the antenna with optional monitoring of antenna temperature.

In other embodiments, a treatment period may be divided into any suitable number of flow periods. In some embodiments, the controller instructs the pump to provide a different constant flow rate for each of the flow periods. In some embodiments, the controller instructs the pump to provide a different variable flow rate for each of the flow periods. For example, a period of low and increasing flow rate may be followed by a period of high and increasing flow rate. In some embodiments, the flow rate may be zero or near-zero for at least part of the treatment period.

The combination of modulated flow rate, cooling lumen geometry and direction of flow provide variables to allow distortions of a microwave antenna system which may not just be limited to achieving a single goal of circular or spherical ablation zones.

In some circumstances, the anatomy of a diseased tissue and or adjacent healthy tissue may warrant an alternative ablation volume. A shorter treatment duration may be used in time critical environments.

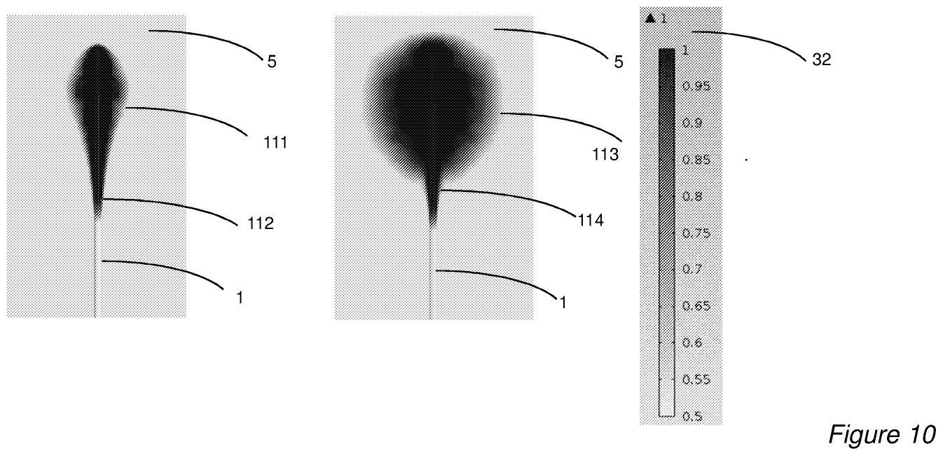

An example of flow rate influencing the necrosis zone that are not entirely circular are given in FIG. 10. Coolant is pumped in through an outer jacket-port into the outer lumen 12 and returns through the inner lumen 11. An initial flow rate of 4.3 ml/min (V=0.2 m/s) is used for the first minute (left plot of FIG. 10). An elongated lachrymiform 112 is formed, extending away from the main necrosis zone 111, in the first minute with a low flow rate as shown in the left plot of FIG. 10. Subsequently a medium flow rate (38.2 ml/min, V=1.78 m/s) is used for the rest of a treatment may be desired (in this case, a remaining 9 minutes). The result of the medium flow rate is shown in the right plot of FIG. 10. The medium flow forms a larger volume 113 which retains the extension 114 formed earlier in the process. In some circumstances, this may have benefit in fully ablating the initial insertion tract to prevent tumour seeding of under treated tissues upon withdrawal of the applicator.

In general, ablation in different shapes may be achieved by modulating the time division (for example 10%/90% or 20%/80% or any other ratio) and/or by modulating the flow rates (lower/higher) and/or by changing diameters of the cooling channels.

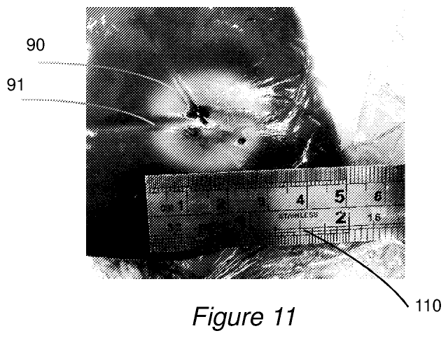

Examples of flow influencing the ablation region in ex-vivo bovine liver are shown in FIG. 11 to FIG. 13. The embodiment used in FIG. 5 of where a high flow rate is used initially for a long duration is shown in ex-vivo tissue in FIG. 11 where the main necrosis region 90 is interrupted by an under treated region 91 where the cooling lumen enters the main treatment zone. The ablation was carried out with a setting of 70 W power at the generator and operated at 5 minutes with a flow rate of 110 mL/min. A linear scale 110 is used to illustrate the magnitude of the distortion. The diagonal line from the top in FIG. 11 is the tract of a thermocouple and not a feature of the embodiment.

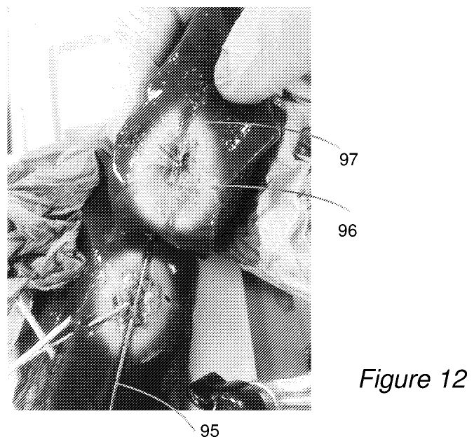

Operating at a higher power than the FIG. 11 embodiment, FIG. 12 shows two halves of the ablation zone 96 created by 100 W of power and operated at 5 minutes with a flow rate of 110 mL/min. The lachrymiform feature 97 becomes slightly more apparent however is much less pronounced to that of lachrymiform feature 22 in FIG. 3.

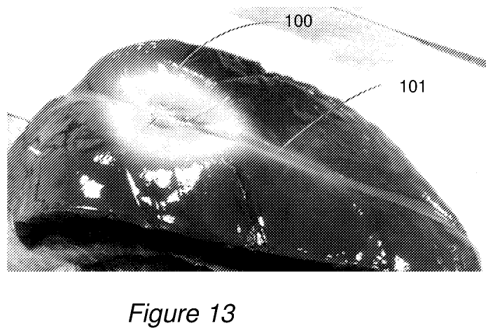

In order to form a more typical cross section of ablation without the under-treated region 91, the power and duration used in the embodiment of FIG. 11 was modulated to a reduced flow rate of 12 mL/min as per FIG. 13. Whilst the main lachrymiform ablation form 100 is apparent in FIG. 13, the under treated region 91 is resolved to become region 101 which has ablated the tract. The method herein can be optimised to produce ablation zones closer to what the theory predicts as desired.

In embodiments described above, the lumens are concentric cylinders that surround the coaxial cable. In further embodiments, a different arrangement of lumens may be used. In some embodiments, multiple individual lumens are arranged around the antenna, for example forming a ring of lumens around a circumference. The controller may control flow of coolant through the multiple lumens to provide an asymmetric cooling effect. For example, the controller may use a higher flow rate in some lumens than in others, thereby providing more cooling in some lumens than in others. One side of the antenna may be cooled more than the other.

The controller may change a path through which the coolant flows, for example sending the coolant through different ones of the lumens. The controller may change flow rate individually in different lumens. In some circumstances, the overall flow rate through the cooling structure may be constant, but the flow rate through individual lumens may change.

Different geometries (for example, different lumen diameters) may result in different flow velocities for the same flow rate of coolant.

In some circumstances, an asymmetric treatment volume may be produced. The asymmetric treatment volume may allow sensitive tissues to be avoided.

In some circumstances, asymmetric cooling may be used to obtain a symmetric treatment volume. For example, the treatment volume may comprise different tissue types, and different degrees of heating may be used for the different tissue types.

In the above embodiments, coolant flow rate is controlled while the power supplied to the antenna is kept constant. In other embodiments, the controller 115 is configured to change the power supplied to the antenna during the treatment. Changes in power supplied may also affect a shape of a treatment volume provided by the antenna.

In some embodiments, the apparatus further comprises at least one temperature sensor. The at least one temperature sensor is configured to measure a temperature in or near the tissue to be heated. In one embodiment, temperature sensors are placed along the length of the antenna shaft 13 and are used to monitor temperature. Signals from the temperature sensors are sent to the controller 115 (or, in other embodiment, to any suitable controller or controllers). The controller 115 controls the flow rate and/or flow direction of the coolant in dependence on the signals from the temperature sensors. The controller 115 may also control a power of the antenna in dependence on the signals from the temperature sensors. Using temperature sensors may allow information to be acquired about the heating effect of the antenna in real tissue, which may be heterogeneous.

In some embodiments, the controller may change a temperature of the coolant being introduced into the cooling lumens.

Embodiments are described above with reference to a monopole antenna. In other embodiments, a cooling system and method similar to that described above may be provided for any suitable antenna, for example a slot, monopole, dipole or triaxial antenna. Any suitable lumen arrangement may be used. A flow rate through at least one lumen may be used to provide a desired shape of a treatment volume.

Embodiments above are described with reference to ablation and tissue necrosis. In other embodiments, the antenna does not perform ablation. The antenna may perform any desired tissue heating process. For example, the antenna may provide more mild temperature elevation than may be used for an ablation process. The more mild temperature elevation may be used for hyperthermia. In some circumstances, temperatures used for surface applications may be lower than temperatures used for penetration applications.

Whether ablation or hyperthermic treatment is performed may be dependent on energy dose. A more dense energy dose may result in heating tissue to a hotter temperature and/or heating tissue more quickly. In some circumstances, a desired result of heating may be cell death. In some circumstances, a desired result of heating may be a call heat reaction, which may not comprise cell death. Parameters (for example, parameters of the antenna and/or of the energy supplied to the antenna) may be selected in order to obtain a desired result of heating.

Embodiments may be used for any appropriate process involving microwave ablation or heating (for example, hyperthermia) of human or animal tissue. The microwave ablation or heating may be performed on any human or animal subject.

In some embodiments, the antenna is introduced into the body of a patient or other subject via a catheter or trocar. In such embodiments, a diameter of the coaxial cable may be such that the antenna can fit into the catheter or trocar used. For example, different catheter sizes may be used for catheters entering different parts of the body. A diameter of the coaxial cable may be appropriate to a diameter of a body part into which the coaxial cable is to be inserted by catheter. The catheter may deliver the antenna to a position adjacent to tissue within the patient or subject, for example to the liver, heart, pancreas, or other organ.

It will be understood that the present invention has been described above purely by way of example, and modifications of detail can be made within the scope of the invention. Each feature disclosed in the description, and (where appropriate) the claims and drawings may be provided independently or in any appropriate combination.

1-33. (canceled)

34. A microwave antenna apparatus comprising:
a radiating element configured to perform a treatment, the treatment comprising heating a volume of tissue using microwave radiation emitted from the radiating element;
a cooling structure arranged for flowing a coolant through at least one lumen of the cooling structure during the treatment; and
a controller configured to control the flow of coolant such as to provide a first flow rate of coolant through the at least one lumen during a first period of the treatment and to provide a second, different flow rate of coolant through the at least one lumen during a second, later period of the treatment.

35. The apparatus according to claim 34, wherein the controller is configured to control the flow of coolant such as to shape the volume of tissue that is heated by the radiating element during the treatment.

36. The apparatus according to claim 34, wherein the controller is configured to control the flow of coolant such as to form in the tissue at least one of a) to d):
a) a spherical treatment zone;
b) a hybrid spherical lachrymiform treatment zone;
c) a partial lachrymiform treatment zone;
d) a partial spherical treatment zone.

37. The apparatus according to claim 34, wherein the controller is configured to control the flow of coolant such that the first flow rate during the first period is higher than the second flow rate during the second period.

38. The apparatus according to claim 34, wherein the controller is configured to control the flow of coolant such that the first flow rate during the first period is lower than the second flow rate during the second period.

39. The apparatus according to claim 34, wherein the first period is longer than the second period.

40. The apparatus according to claim 34, wherein the second period is longer than the first period.

41. The apparatus according to claim 34, wherein at least one of a), b) or c):
a) the controller is configured to vary the flow rate continuously over time during the treatment.
b) the controller is further configured to alter a power of the radiation emitted from the radiating element during the treatment; or
c) the controller is configured to control the power of the radiation such as to shape the volume of tissue that is heated by the radiating element during the treatment.

42. The apparatus according to claim 34, wherein the cooling structure comprises at least one inner lumen positioned radially proximal to the radiating element and at least one outer lumen positioned radially distal to the radiating element, and wherein either a) or b):
a) the controller is configured to control the flow of coolant such that the coolant flows into the at least one inner lumen and returns through the at least one outer lumen, or
b) the controller is configured to control the flow of coolant such that the coolant flows into the at least one outer lumen and returns through the at least one outer lumen.

43. The apparatus according to claim 34, wherein the cooling structure comprises a plurality of lumens arranged circumferentially around the radiating element.

44. The apparatus according to claim 43, wherein at least one of
a) or b):
a) the controller is configured to select lumens through which the coolant flows in and out such as to shape the volume of tissue that is heated by the radiating element during the treatment; or
b) at least one diameter of at least one lumen is selected such as to shape the volume of tissue that is heated by the radiating element during the treatment.

45. The apparatus according to claim 35 wherein at least one of a) or b), wherein:
a) the shape of the volume of tissue that is heated is controlled so as to reduce or eliminate heating of an anatomical structure, optionally wherein the anatomical structure comprises at least one natural lumen; or
b) the shape of the volume of tissue that is heated is controlled to reduce tissue shrinkage in the vicinity of the radiating element.

46. The apparatus according to claim 34, further comprising at least one temperature sensor configured to monitor temperature within the volume of tissue to be heated, wherein the controller is configured to control the flow of coolant in dependence on signals from the at least one temperature sensor, wherein the signals are representative of the monitored temperature.

47. The apparatus according to claim 46, wherein the controller is further configured to control in dependence on the signals from the at least one temperature sensor at least one of: a power supplied to the radiating element, a direction of coolant flow, or a coolant flow path.

48. The apparatus according to claim 34, wherein at least one of a), b), c), d), e) or f), wherein:
a) the radiating element is formed from coaxial cable;
b) the radiating element comprises at least one of a slot antenna, a monopole antenna, a dipole antenna, a triaxial antenna;
c) the coolant comprises at least one of a gas coolant, a liquid coolant, air, saline
d) the apparatus is configured to perform microwave ablation of tissue and/or tissue hyperthermia;
e) the flowing of the coolant through at least one lumen of the cooling structure is to cool the radiating element and/or to cool a cable supplying power to the radiating element; or
f) the radiating element and the cooling structure are housed within a common housing.

49. A microwave system comprising:
a microwave generator;
a microwave cable apparatus comprising a coaxial cable, wherein an exposed distal portion of an inner conductor of the coaxial cable is longer than an outer conductor of the coaxial cable, the exposed distal portion forming a radiating element, wherein the radiating element is configured to perform a treatment, the treatment comprising heating a volume of tissue using microwave radiation emitted from the radiating element;
a cooling structure arranged for flowing a coolant through at least one lumen of the cooling structure during the treatment; and
a controller configured to control the flow of coolant such as to provide a first flow rate of coolant through the at least one lumen during a first period of the treatment and to provide a second, different flow rate of coolant through the at least one lumen during a second, later period of the treatment.

50. A method comprising:
performing a treatment comprising heating a volume of tissue using microwave radiation emitted from a radiating element;
during the treatment, flowing a coolant through at least one lumen of a cooling structure; and
controlling the flow of coolant such as to provide a first flow rate of coolant through the at least one lumen during a first period of the treatment and to provide a second, different flow rate of coolant through the at least one lumen during a second, later period of the treatment.

51. The method according to claim 50, wherein the controller is configured to control the flow of coolant such as to shape the volume of tissue that is heated by the radiating element during the treatment.

52. A method comprising:
receiving parameters of a radiating element for emission of microwave radiation and a cooling structure comprising at least one lumen;
receiving a desired volume of tissue to be heated by the emission of microwave radiation from the radiating element; and
determining a first flow rate of coolant and second flow rate of coolant to be provided through the at least one lumen to shape a volume of tissue heated by the emission of microwave radiation by the radiating element to match the desired volume of tissue, wherein the determining is in dependence on the parameters of the radiating element and the cooling structure.

52. The method according to claim 52, wherein at least one of a) b), c), d), e) or f), wherein:
a) the method further comprises determining a direction of coolant flow through the at least one lumen;
b) the method further comprises determining a path of coolant flow;
c) the method further comprises determining a power to be provided to the radiating element;
d) the method further comprises determining a period of time over which the first flow rate is to be delivered;
e) the method further comprises determining a period of time over which the second flow rate is to be delivered
f) the determining is further in dependence on at least one temperature measurement for tissue heated by the radiating element.
